(19) United States
(12) Patent Application Publication
(10) Pub. No.: US 2006/0079753 A1
(43) Pub. Date: Apr. 13, 2006

(54) Pre-acquisition identification of region for image acquisition time optimization for radiation imaging systems

(76) Inventors: Michael Gurley, San Diego, CA (US); Mark Harpin, San Diego, CA (US); Samia Arram, San Diego, CA (US)

Correspondence Address: FISH & RICHARDSON, PC, P.O. BOX 1022, MINNEAPOLIS, MN, 55440-1022, US

(21) Appl. No.: 11/238,161

(22) Filed: Sep. 27, 2005

Related U.S. Application Data
(60) Provisional application No. 60/613,781, filed on Sep. 27, 2004.

(51) Int. Cl. A61B 5/05

(52) U.S. Cl. 600/407

(57) ABSTRACT

A SPECT imaging system has at least one detector head, adapted for detecting a radioisotope emission from a patient, and a collimator, which directs said radioisotope emission towards said detector head, a movement subsystem, which moves at least one of a patient being imaged, or said detector heads, relative to the other. A controlling computer, includes a user interface receiving output from the at least one detector head, and controls said movement subsystem, said controlling computer including a user interface, receiving information said output from the detector head, and controls at least one parameter associated with the reception of information by the detector head. The controller displays information about a region of interest within a image to be determined, and automatically determines at least one recommended parameter for the scan based on said region of interest.

### CLAIM OF PRIORITY

This application claims priority from provisional application Ser. No. 60/613,781, filed Sep. 27, 2004.

### BACKGROUND

Medical imaging systems may use nuclear materials, called radiopharmaceuticals, for the imaging. One such imaging system is single photon emission computed tomography, abbreviated as SPECT. Other techniques may include other kinds of nuclear medicine, positron emission tomography (“PET”) as well as magnetic resonance imaging.

Imaging systems of this type may be dependent on many variables including, but not limited to, characteristics of the specific patient, also called patient demographics, selection of the collimator which is used for the photon emission, the kind of radiation detector which is used, and the uptake of the radiopharmaceutical in the patient. Exemplary patient demographics that may lead to image inconsistencies may include patient variables such as patient size and weight, as well as normal differences between the locations of organs in different people. The different ways in which these variables are carried out may affect the image quality that is achieved in the nuclear medical image acquisition.

Importantly, the variation in image quality may result itself in quality variations that may lead to inconsistencies in interpretation. Any such inconsistencies may be undesirable, since they may reduce the confidence in image interpretation. Attempts have been made to improve the consistency of the images. However, these improvement attempts are often relatively slow, and may interrupt the flow and workload of the organization that obtains the images.

The American Society of Nuclear Cardiology (ASNC), the largest Nuclear Cardiology organization, developed and approved imaging guidelines, entitled “Imaging Guidelines for Nuclear Cardiology Procedures, Part 1”, Revised in 2000. These were published as “Updated Imaging Guidelines for Nuclear Cardiology Procedures, Part 1” (Journal of Nuclear Medicine, January/February 2001, part 1, volume 8, number 1). These guidelines are designed to help standardize SPECT acquisition protocols. These protocols pertain directly to recommended acquisition times and procedures, describing the desired minimum and maximum number of “counts” accumulated in the desired area of imaging, e.g., the heart. The “counts” refer to the amount of detected emission from the heart, responsive to the incoming radiation. In one embodiment, the incoming radiation may be gamma radiation.

One guideline is used for determining if the imaging of the heart has received enough “counts” of gamma radiation, that is, is the heart “count sufficient” or “count starved”. However, the practical implementation of these guidelines suggests that the operators of the imaging system perform manual calculations. Because of the additional time needed to perform these calculations and the corresponding effect on throughput of the imaging process, many operators have opted not to use them.

Other, less precise indicia, such as generic acquisition time recommendations are often used. These indicia do not account for important variables such as patient size and or weight.

### SUMMARY

The present application teaches techniques to identify a region of interest, within an image to be scanned, prior to acquisition of that image. In an embodiment, the region of interest is identified within an image that represents the human body part or area, of which an image will be obtained. An aspect allows using a user interface to interact with and /or change the region of interest, and to change characteristics of the eventual scanning.

### BRIEF DESCRIPTION OF THE DRAWINGS

These and other aspects will now be described in detail with reference to the accompanying drawings, wherein:

FIG. 1 shows a persistence display of a patient, including the projection image of an organ of interest, and an exemplary box shown as drawn around that organ of interest;

FIG. 2 shows a user interface display which may display information to a user;

FIG. 3 illustrates a block diagram of the controlling system; and

FIG. 4 shows a flowchart of operation.

### DETAILED DESCRIPTION

The general structure and techniques, and more specific embodiments which can be used to effect different ways of carrying out the more general goals are described herein.

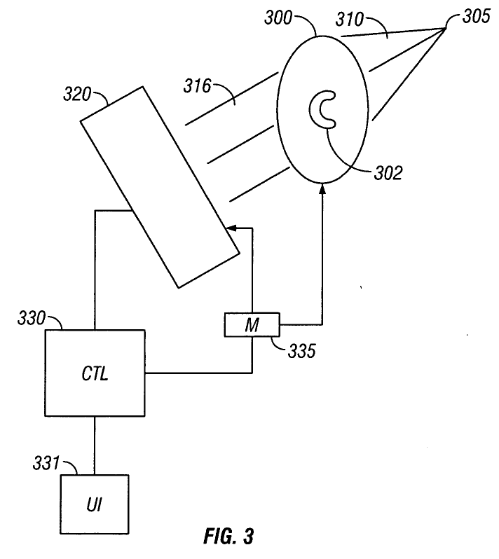

One embodiment describes identifying a region of interest within an image to be scanned prior to its acquisition. FIG. 3 illustrates an exemplary system. A patient, shown as 300, is illustrated along with an organ of interest which may be the heart. A radiopharmaceutical, e.g., a radioisotope 305 is within the patient body. A collimator 305 is used to focus the emission along a path 310 as a radiation beam. The emission is detected by a detector head 320, so long as the detector head 320 is along the path 310 formed by the collimator. If parallel-beam collimators are used, the path 310 should be perpendicular to the surface of the detector head 320 surface. Of course, other collimator types may also be used. The beam 31 is collected by at least one detector head 320 after passage through the patient 300. While the above has described an emission study type image, it should be understood that this can also be applied to transmission type studies.

The output of the detector head 320 is processed by a processing element/controller 330. The processing element may be a computer. This may be any kind of computer, either general purpose, or some specific purpose computer such as a workstation. The computer may be a Pentium class computer, running Windows XP or Linux, or may be a McIntosh computer. The programs may be written in C, or Java, or any other programming language. The programs may be resident on a storage medium, e.g., magnetic or optical, e.g. the computer hard drive, a removable disk or other removable medium. The programs may also be run over a network.

The processing element may also control the movement of at least one of the detector head 320, or the patient 300 using a movement subsystem 335. For example, either the patient 300 may be rotated or the heads may be rotated to receive information from the patient.

In an embodiment, the controlling unit 330 may include the ability to identify a region of interest in the image to be scanned prior to its acquisition. The techniques for doing this may be carried out in software, firmware or hardware. The controller 330 may include a general-purpose processor, such as an Intel Pentium class processor, or any other type of processor as may be understood by those having ordinary skill in the art. The controller 330 may have an associated user interface 331.

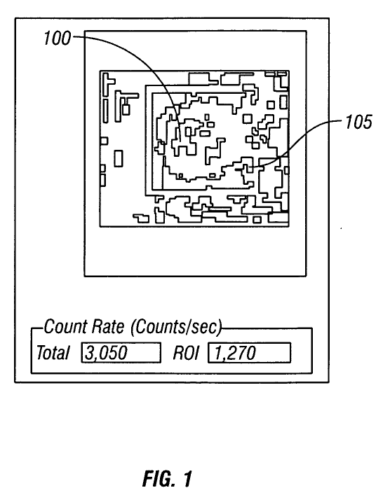

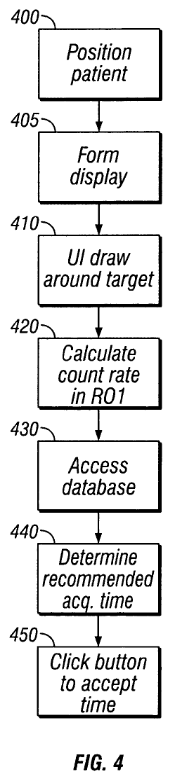

In operation, the controller may run a routine shown in the flowchart of FIG. 4 prior to image acquisition time. A region of the patient is in a specific condition at that time, for example the region may be resting or under stress, and the routine may use a particular radiation imaging system. In the embodiment, the system may be a gamma ray imaging system used to generate single photon emission computed tomography images. At 400, the imaging target, typically the patient is positioned into the specific location where the patient will be imaged. A display on the user interface 331 is formed at 405; for example, a persistence display that shows the imaging target. The display may be formed from an initial medical imaging scan of the patient, using an emission or transmission scan, an xray scan, MRI or any other technique. At 410, the user interface is used to draw a rectangle around the display of the region of interest. The operator can readily identify the scan, thereby facilitating the drawing. For example, the computer mouse or other pointing device can be used for this purpose. The embodiment, as shown in FIG. 1, draws a corresponding rectangle on the display, surrounding the target.

In another embodiment, other types of user interface devices may be used to draw on the target. For example, a pointing device may be used to point directly at the target, draw a circle around the target, or may be used with other ways of identifying the region of interest. Using conventional techniques, the image within that region of interest may be smoothed and reshaped.

According to another embodiment, an image processing system may include kernels indicative of usual shapes of regions of interest at display 405. For example, a database of usual heart shapes in the display 405 may be maintained, and correlated against the image in FIG. 1. This correlation may be used to automatically identify the heart in the display, as the region of interest. By actually selecting the region of interest, image inconsistencies may be reduced.

At 420, the count rate within the identified region of interest is calculated. The count rate within the entire field of view may also be calculated.

At 430, a database of common imaging targets is accessed. This database includes information about imaging targets such as the heart, liver, bone, and other targets. Based on the information from the database, the embodiment may then determine a recommended acquisition time at 440. For example, this may be done by using a local table that correlates the organs to the acquisition time calculation. Alternatively, the computer may calculate the information, using either a formula, or model, or any other technique. It may use any other type of data detection and/or analysis system.

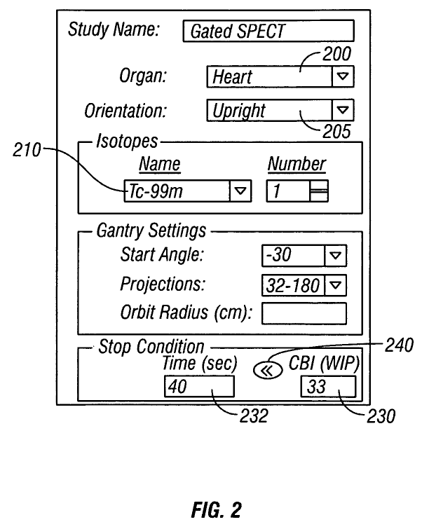

The selected working, as well as the count rate in the region of interest, are used to display a recommended acquisition time. FIG. 2 illustrates an exemplary dialog box that shows the organ, as well as different information about the display. In FIG. 2, the organ is shown as 200, and the orientation shown as 205. The measuring isotope, here tc-99m is selected. The entry settings and projections may also be analogously selected. The system then displays this recommended time at 230 which may be used for the imaging. The operator may choose to adopt the recommendation, or alternatively may choose to ignore it. If the operator chooses to adopt the recommendation, of that time can be automatically accepted by clicking the button 240 on the display. FIG. 2 illustrates an exemplary stop condition window for an exemplary heart image acquisition 232 represents the stop condition initially entered by the operator, of 40 seconds. The calculated stop condition at 230 is shown as 33 seconds. The operator can then click a button 232 to copy the entry in the recommended time box into the final value. The user can then proceed with the acquisition using the recommended time. 450 illustrates the user clicking the button to accept the recommended time.

This embodiment allows count rate and count density on a persistence mode display to be used as a basis for quantified quality control prior to the actual image acquisition. Unlike post-acquisition processes, this system may enhance compliance with guidelines without significantly compromising the throughput of patients through the imaging process. Moreover, the techniques disclosed herein may be used along with a post-acquisition tool, and also may be used with other kinds of imaging that are used in place of or in addition to the SPECT imaging.

Although only a few embodiments have been disclosed in detail above, other embodiments are possible and the inventor(s) intend these to be encompassed within this specification. The specification describes specific examples to accomplish a more general goal that may be accomplished in other way. This disclosure is intended to be exemplary, and the claims are intended to cover any modification or alternative which might be predictable to a person having ordinary skill in the art. For example, this may be used with different kinds of imaging than that disclosed; it may be used with multiple different imaging techniques, and it may be used with other ways of selecting the area of interest, including automatic techniques of detecting the area of interest. One exemplary way of determining the region of interest is to store a number of image “kernels”, each kernel representing an exemplary view of a specified organ. For example, views of the most common kinds of organs can be stored. The way the heart looks from many different directions and/or in many different patients and/or with many different machines can be stored. Each of the kernels may be correlated over the entire image, using least mean squares matching, to find the closest match to the kernels. For example, if a close match to the heart kernel is found, then the area of that match is determined to be a heart, and may be automatically outlined by the computer as the region of interest.

While the above describes a single detector head, it should also be understood that there can be multiple separated detector heads.

Also, the inventors intend that only those claims which use the words “means for” are intended to be interpreted under 35 USC 112, sixth paragraph. Moreover, no limitations from the specification are intended to be read into any claims, unless those limitations are expressly included in the claims.

What is claimed is:

1. A method, comprising:
obtaining initial information about a region of interest to be imaged within a patient;
using a computer to automatically determine a recommended parameter for said region of interest using a specified medical imaging type; and
using said recommended parameter as a part of said specified medical imaging.

2. A method as in claim 1, wherein said obtaining comprises first, displaying an image representing an initial view of a medical scan of a patient; and then enabling selecting a portion of said view as a region of interest.

3. A method as in claim 1, wherein said specified scanning type is a positron emission.

4. A method as in claim 3, wherein said recommended parameter includes a time of scanning.

5. A method as in claim 3, wherein said time of scanning is selected as a time that causes a specified number of counts of radioisotope emissions to be acquired from said region of interest.

6. A method as in claim 1, wherein said enabling selecting comprises enabling a manual selection of said portion as said region of interest.

7. A method as in claim 1, wherein said obtaining comprises automatic selection of a specified body part as said region of interest.

8. A method as in claim 5, wherein said obtaining comprises determining an expected count rate within the region of interest.

9. A method as in claim 8, wherein said determining comprises accessing a database to determine said region of interest.

10. A method as in claim 1, wherein said using comprises displaying said recommended parameter, and enabling accepting said recommended parameter.

11. A method as in claim 1, wherein said medical scan is a SPECT scan.

12. An apparatus, comprising:
a detector head, adapted for receiving emissions from a radiopharmaceutical source and producing at least one output indicative of said emissions;
a controller, receiving said output from the detector head, and controlling at least one parameter associated with said receiving by the detector head, said controller enabling information about a region of interest within a medical image to be determined, and automatically determining at least one recommended parameter for said medical image based on said region of interest.

13. An apparatus as in claim 12, wherein said controller operates to produce a display indicative of a scan, enabling selection of a region of interest within said scan, and determine said parameters based on said selecting.

14. An apparatus as in claim 12, wherein said controller accepts manual input indicative of a perimeter of said region of interest.

15. An apparatus as in claim 12, wherein said controller automatically determines said region of interest.

16. An apparatus as in claim 12, wherein said parameter includes a time of scanning.

17. An apparatus as in claim 12, wherein said parameter is calculated by said controller.

18. An apparatus as in claim 12, wherein said controller stores at least one table which correlates image information to recommended parameters, and said controller operates to look up said parameter in said table.

19. An apparatus as in claim 12, further comprising a movement subsystem which moves at least one of the patient and/or the detector head relative to one another.

20. An apparatus as in claim 12, wherein said controller calculates said parameter based on an expected count rate within a specified region of interest.

21. A method, comprising:
first, forming an initial view of the display representing a medical scan within a patient;
enabling a portion of the display which represents a specified region of interest for a subsequent medical scan to be determined;
determining parameters for a desired radioisotope count rate within said region of interest; and
using said parameters for said subsequent medical scan.

22. A method as in claim 21, wherein said enabling determining the portion of the display comprises automatically determining a region of interest within the display.

23. A method as in claim 21, wherein said enabling determining the portion of the display comprises enabling manual selection of a perimeter of the region of interest.

24. A method as in claim 21 wherein said determining parameters comprises accessing a database to determine said parameters.

25. An apparatus, comprising:
at least one detector head, adapted for detecting a radioisotope emission from a patient;
a collimator, which directs said radioisotope emission towards said detector head;
a movement subsystem, which moves at least one of a patient being imaged, or said detector heads, relative to the other; and
a controlling computer, including a user interface receiving output from said at least one detector head, and controlling said movement subsystem, said controlling computer including a user interface, receiving information said output from the detector head, and controlling at least one parameter associated with the reception of information by the detector head, said controller displaying information about a region of interest within a image to be determined, and automatically determining at least one recommended parameter for said scan based on said region of interest.
